(19) United States
(12) Patent Application Publication
(10) Pub. No.: US 2024/0138940 A1
(43) Pub. Date: May 2, 2024

(54) SURGICAL ROBOTIC SYSTEM AND METHOD FOR USING INSTRUMENTS IN TRAINING AND SURGICAL MODES

(71) Applicant: Covidien LP, Mansfield, MA (US)

(72) Inventors: Emily M. Ludwig, New Haven, CT (US); Brian A. Rockrohr, Guilford, CT (US)

(21) Appl. No.: 18/383,186

(22) Filed: Oct. 24, 2023

Related U.S. Application Data
(60) Provisional application No. 63/420,154, filed on Oct. 28, 2022.

(51) Int. Cl. A61B 34/32; G16H 40/63

(52) U.S. Cl. CPC A61B 34/32; G16H 40/63; A61B 2090/0803

(57) ABSTRACT

A surgical robotic system includes a surgical instrument and a robotic arm having an instrument drive unit configured to couple to and to actuate the surgical instrument. The system also includes a controller configured to: access usage data pertaining the surgical instrument; select an instrument operational mode for the surgical instrument based the usage data, the instrument operational mode being one of a surgical mode or a training mode; and enable use of the surgical instrument based on the selected instrument operational mode.

### CROSS-REFERENCE TO RELATED APPLICATIONS

The present application claims the benefit of and priority to U.S. Provisional Patent Application Ser. No. 63/420,154, filed on Oct. 28, 2022, the entire contents of which being incorporated herein by reference.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Robotic surgery is complex and requires training and/or demonstrations to new users. However, surgical instruments are expensive and have limited usability in surgical procedures making training using actual instruments expensive.

### SUMMARY

Surgical instruments have stringent reliability requirements, in place to ensure patient safety. Consequently, surgical instruments with limited reuse may functionally have significant useful life remaining even after the instrument is past its clinical use expiration point. This usage data can be harnessed for other purposes, such as training and demonstration.

This disclosure focusses on these extended uses, and how an intelligent system, such as a robotically assisted surgical system can enable these additional use cases. Surgical instruments with limited reuse may be able track usage—either directly on the instrument, or through a system of which the instrument is a part. This usage tracking may be used to disable an instrument and/or prevent subsequent usage once the tracked usage exceeds a pre-determined threshold. Typically, the instrument would then be disposed of, as there would be no further use for an expired instrument.

While usage limits are critical to ensure patient safety, expired instruments may have significant usable usage remaining between when they expire and when they fail to be functionally effective. This remaining instrument usable usage can be captured for purposes other than clinical use, for example in training (such as for animal or cadaveric use), skills development (such as to practice the fundamentals of robotic surgery), or for demonstration of system functionality, etc.

This additional value can be delivered through a variety of implementations, such as toggling an indicator in the instrument memory once a usage threshold has been passed. This indicator is then used to ensure that the system only allows attachment and continued use of the instrument if the system is configured in a training or demonstration mode.

Setting the instrument into the training/demonstration mode could have additional effects, such as allowing the system to derate the instrument performance, thereby further extending the useful life of the instrument. Performance derating may include operating an instrument drive unit, and by extension the instrument at reduced clamp forces, reduced stiffness in articulation degrees of freedom, slower accelerations, etc.

According to one embodiment of the present disclosure, a surgical robotic system is disclosed. The surgical robotic system includes a surgical instrument and a robotic arm having an instrument drive unit configured to couple to and to actuate the surgical instrument. The system also includes a controller configured to: access usage data pertaining to the surgical instrument; select an instrument operational mode for the surgical instrument based the usage data, the instrument operational mode being one of a surgical mode or a training mode; and enable use of the surgical instrument based on the selected instrument operational mode.

Implementations of the above embodiment may include one or more of the following features. According to one aspect of the above embodiment, in the surgical operation mode, the controller may be further configured to fully power the instrument drive unit. In the training mode, the instrument drive unit may be partially powered. The controller may be further configured to determine whether the surgical instrument is expired based on the usage data, and prevent use of the surgical instrument in response to the determination. The controller may be further configured to update the usage data following the use of the surgical instrument in the selected instrument operational mode. The controller may be also configured to receive a system operational mode (e.g., from a user or from the system itself) which may be one of the surgical mode or the training mode. The controller may be additionally configured to enable use of the surgical instrument based on the selected instrument operational mode and the system operational mode. The usage data may include at least one of number of activations, number of uses, total force, total torque, time used, or a mode indicator.

According to another embodiment of the present disclosure, a surgical robotic system is disclosed. The surgical robotic system includes a surgical instrument and a robotic arm having an instrument drive unit configured to couple to and to actuate the surgical instrument. The system also includes a controller configured to: set a system operational mode of the robotic arm in one of a surgical mode or a demonstration mode; access usage data pertaining the surgical instrument; compare the usage data to a usage threshold corresponding to an instrument operational mode, where the instrument operational mode is one of the surgical mode or the demonstration mode; prevent use of the surgical instrument in response to the usage data being below the usage threshold; and enable use of the surgical instrument in response to the usage data being above the usage threshold and the system operational mode.

Implementations of the above embodiment may include one or more of the following features. According to one aspect of the above embodiment, in the surgical mode, the controller may be further configured to fully power the instrument drive unit. In the demonstration mode, the instrument drive unit may be partially powered. The controller may be further configured to make a determination as to whether the surgical instrument is expired based on the usage data, and prevent use of the surgical instrument in response to the determination. The controller may be also configured to update the usage data following the use of the surgical instrument. The usage data may include at least one of number of activations, number of uses, total force, total torque, time used, or a mode indicator.

According to a further embodiment of the present disclosure, a method for enabling use of a surgical instrument is disclosed. The method includes accessing usage data pertaining the surgical instrument. The usage data may include usage data of the surgical instrument. The method also includes comparing the usage data to a usage threshold corresponding to an instrument operational mode of a surgical robotic system, where the instrument operational mode is one of a surgical mode or a training mode. The method further includes preventing use of the surgical instrument in response to the usage data being below the usage threshold, and enabling operation of the surgical instrument by an instrument drive unit in response to the usage data being above the usage threshold.

Implementations of the above embodiment may include one or more of the following features. According to one aspect of the above embodiment, the method may further include operating the surgical instrument in the surgical operation mode by fully powering the instrument drive unit. The method may also include operating the surgical instrument in the training mode by partially powering the instrument drive unit. The method may additionally include making a determination as to whether the surgical instrument is expired based on the usage data, and preventing use of the surgical instrument in response to the determination. The method may additionally include updating the usage data following the use of the surgical instrument. Accessing usage data may also include reading at least one of number of activations, number of uses, total force, total torque, time used, or a mode indicator.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical system of FIG. 1 positioned about a surgical table according to an embodiment of the present disclosure;

FIG. 6 is a rear perspective view of a surgical instrument according to an embodiment of the present disclosure; and

FIG. 7 is a flow chart illustrating a method for tracking useful life of instruments and enabling their use in surgical and training modes according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement command s and to generate torque commands for activating one or more actuators of the robotic arm, which would, in turn, move the robotic arm in response to the movement command.

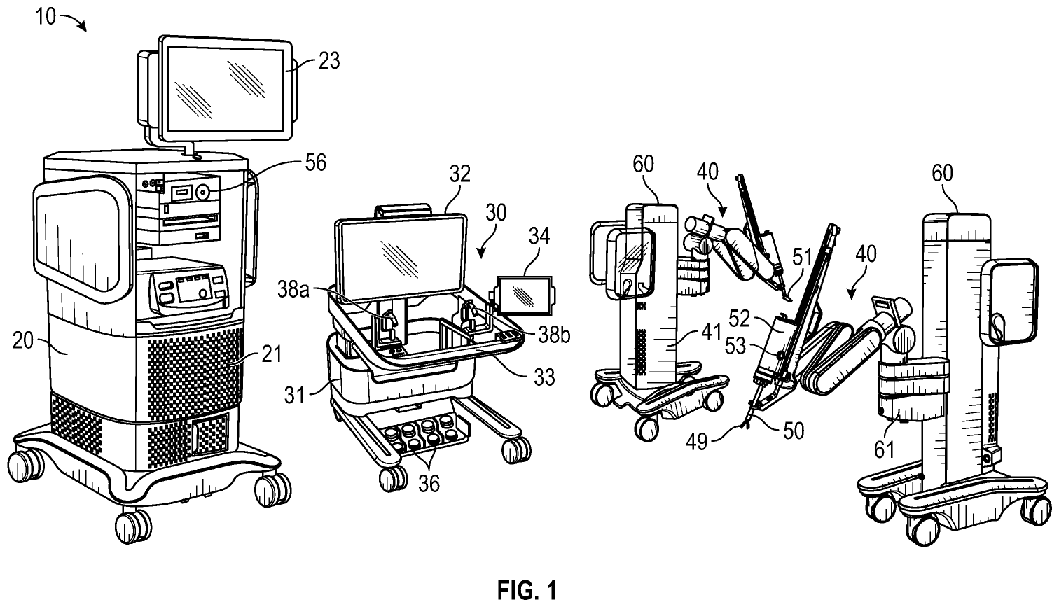

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 removably coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include a laparoscopic camera 51 configured to capture video of the surgical site. The laparoscopic camera 51 may be a stereoscopic endoscope configured to capture two side-by-side (i.e., left and right) images of the surgical site to produce a video stream of the surgical scene. The laparoscopic camera 51 is coupled to a image processing device 56, which may be disposed within the control tower 20. The image processing device 56 may be any computing device as described below configured to receive the video feed from the laparoscopic camera 51 and output the processed video stream.

The surgeon console 30 includes a first screen 32, which displays a video feed of the surgical site provided by camera 51 of the surgical instrument 50 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the TREF 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

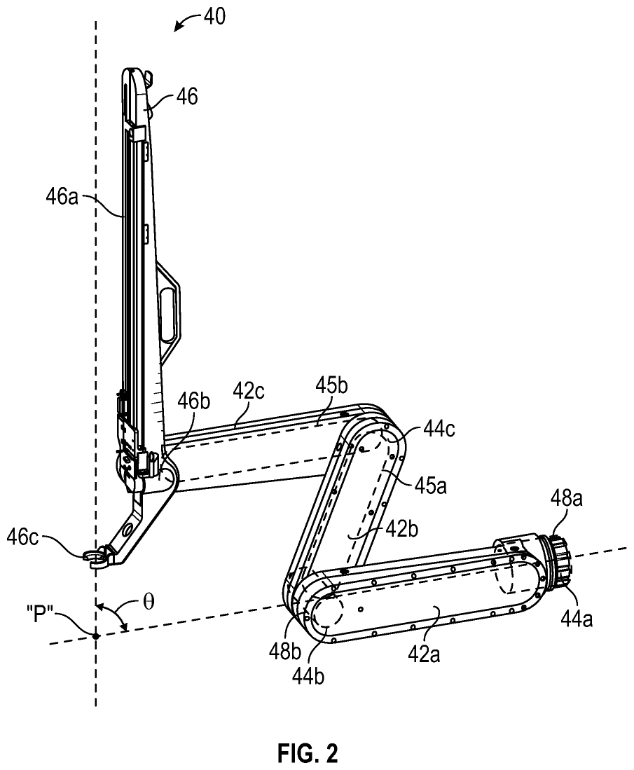

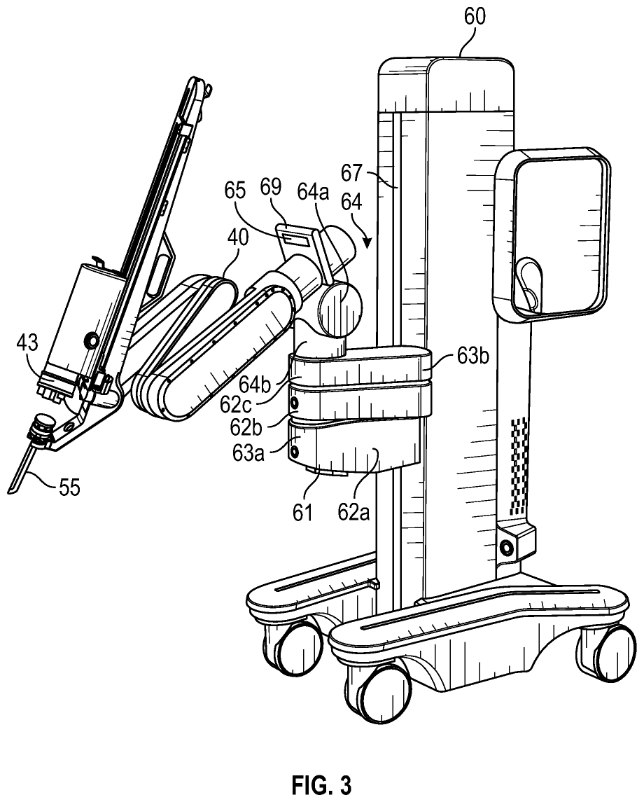

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

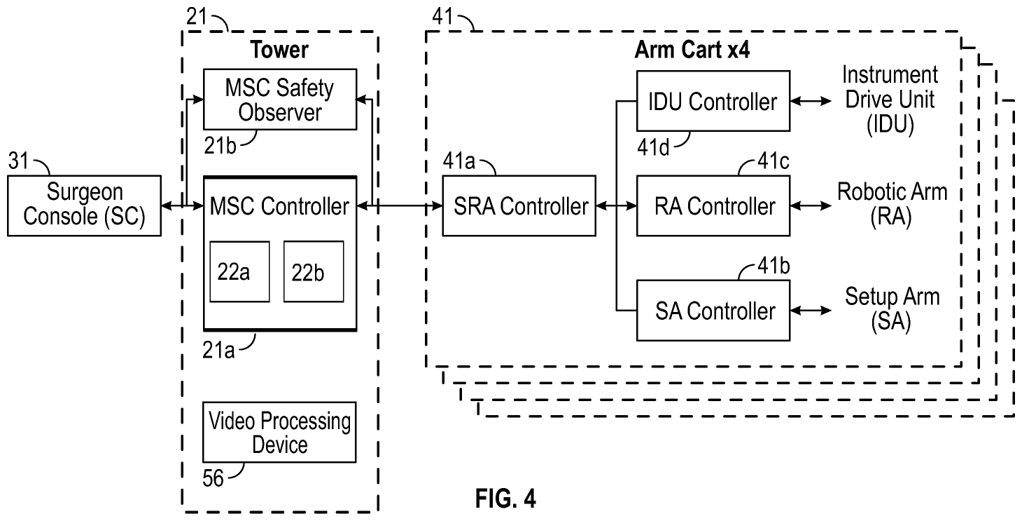

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the hand controller 38a is then scaled by a scaling function executed by the controller 21a. In embodiments, the coordinate position may be scaled down and the orientation may be scaled up by the scaling function. In addition, the controller 21a may also execute a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

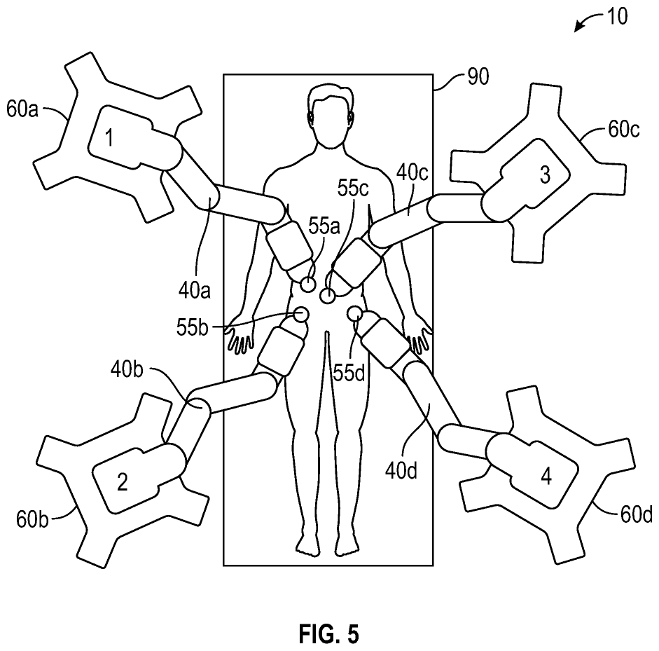

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

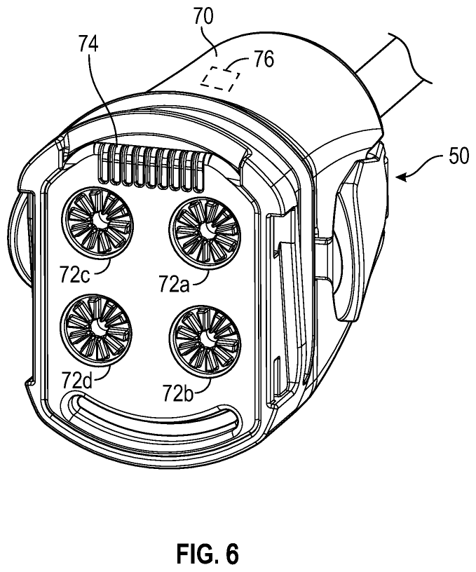

With reference to FIG. 6, the instrument 50 includes a housing assembly 70 enclosing drive couplers 72a-d configured for selective connection to the IDU 52. The IDU 52 includes a plurality of motors configured to actuate each of the drive couplers 72a-d thereby actuating the instrument 50. The housing assembly 70 also includes an electrical connector 74 configured for selective connection to the IDU 52. The instrument 50 may include electronics, including, and not limited to, a memory 76, wired or wireless communication circuitry for receiving and transmitting data or information. The IDU 52 may be configured to permit passage or routing of a dedicated electrocautery cable or the like for use and connection to an electrosurgical based electromechanical surgical instrument (e.g., for ablation, coagulation, sealing, etc.).

The memory 76 may be any suitable storage device, such as flash memory and is configured to store identification information of the instrument 50, usage data, and the like. The memory 76 may be accessed by any controllers of the surgical robotic system 10, which may be the computers 21, 31, 41. In an exemplary embodiment, the main controller 21a is configured to read and write to the memory 76 including retrieving and updating usage data. The computers 21, 31, 41 are configured to communicate with the memory 76 through the electrical connector 74 and/or any other wired or wireless interface.

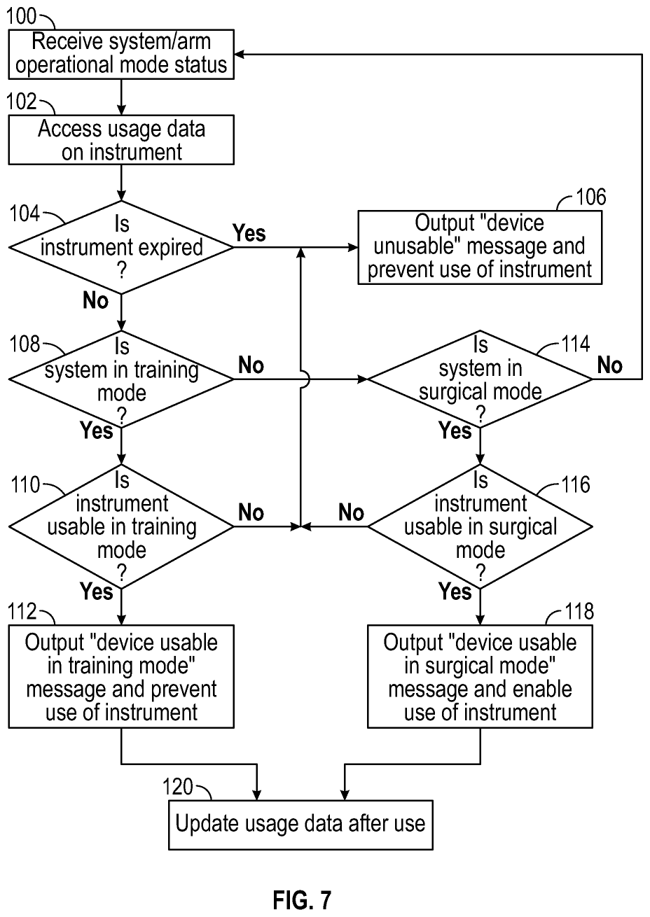

With reference to FIG. 7, a method for operating the system 10 and the instrument 50 in different operational modes is disclosed. The system 10 may be operated in a plurality of operational modes including a surgical mode and a training and/or demonstration operational mode. In the surgical mode, the system 10 is used to perform surgical procedures on a patient, whereas in a training mode (used synonymously with a demonstration, or any non-surgical mode) the system 10 is not used to perform surgical procedures on patients. Training mode may include allowing the user to manipulate the instrument 50 on a cadaver, a training implement, etc. In embodiments, rather than the system 10 switching between different modes, the system 10 may include different hardware or software versions for operating in one of the modes, i.e., surgical or training modes. The system 10 is configured to determine whether the instrument 50 is usable with the system 10 based on the usage data of the instrument 50 and depending on the operational mode of the system 10.

The method of FIG. 7 may be implemented as software instructions executable by the controller 21a or any other processor of the system 10. Initially, at step 100, the controller 21a receives the status of the operational mode to which the system 10 is set. In embodiments, the operational mode to which the system 10 is set may be the default setting, e.g., where the system 10 is configured solely for one of surgical or training use. In other embodiments, the operational mode to which the system 10 is set may be selected by a user (or another, automatic selection mechanism built into the system 10), e.g., where the system 10 is configured for operation in a plurality of modes.

For each instrument 50 that is connected to the robotic arm 40, the controller 21a executes the following use prediction algorithm. At step 102, the usage data from the memory 76 of the instrument 50 is accessed by the controller 21a. Usage data may be stored in any suitable data structure and may include the number of activations or uses, total force or torque applied, actual time used (which may be measured in seconds, minutes), etc. Use of the instrument 50 may constitute coupling the instrument 50 and activating the drive couplers 102a-d by the IDU 52. Thus, once this event occurs, the usage count is incremented by the controller 21a. Maximum life values may be set at the factory for each of the instruments 50.

Usage data also includes remaining life of the instrument 50. In embodiments, the main controller 21a may calculate the remaining life of the instrument 50 based on the usage data and display the remaining life on any of the screens 23, 32, and/or 34. In particular, the controller 21a may calculate remaining instrument life for the instrument 50 based on previous use of the instrument 50. The controller 21a may calculate remaining life as a number of uses remaining and/or a percentage of life remaining based on usage data, e.g., total minutes used vs. the total minutes allowed for the instrument 50.

In addition to usage data, the instrument 50 may also store a mode indicator indicating whether the instrument 50 is usable in a surgical mode, a training mode, or is expired and cannot be used in any mode with the system 10. Furthermore, the instrument 50 that is usable in a surgical mode, may also be enabled for use in training mode. However, the system 10, when in operating in the training mode, may be configured to operate only with instruments 50 that are no longer usable in surgical mode. This may be useful to prevent the waste of surgical instruments 50 that are usable in surgical procedures in training procedures.

At step 104, the controller 21a determines whether the instrument 50 is expired and is thus, unusable by the system 10 in either of the operational modes. This may be determined by comparing the usage data (e.g., time of use, number of uses, etc.) to an expiration threshold indicative of expiration of the useful life of the instrument 50. In further embodiments, the instrument 50 may include a mode indicator indicative that the instrument 50 is expired.

At step 106, after determining that the instrument 50 is expired, the controller 21a outputs a message on one of the screens 32, 34, etc. of the system 10 stating that the instrument 50 is expired or otherwise unusable. The controller 21a also prevents use of the instrument 50 by blocking any user inputs or actions that would activate or pair the instrument 50 to the IDU 52 and the system 10 at large.

After determining that the instrument 50 is not expired, the controller 21a proceeds with determining whether the instrument 50 is usable in one or more of the operational modes of the system 10. At step 108, the controller 21a determines whether the system 10 is in a training mode If so, then the controller 21a determines whether the instrument 50 is usable in a training mode at step 110. This may be determined by comparing the usage data to a training mode threshold or range. If the remaining useful life of the instrument is below the training mode threshold (i.e., remaining useful life is too low), then the instrument 50 is not usable and the controller 21a proceeds to step 106. The controller 21a outputs a message on one of the screens 32, 34, etc. of the system 10 stating that the instrument 50 is expired or otherwise unusable. The controller 21a also prevents use of the instrument 50 by blocking any user inputs or actions that would activate or pair the instrument 50 to the IDU 52 and the system 10 at large.

By way of an example, if the training mode threshold is 40% and the instrument 50 has 50% of useful life left, then the instrument 50 is useful in the training mode. Conversely, if the instrument 50 has only 30% of useful life left, then the instrument 50 is not usable in the training mode. In further embodiments, the instrument 50 may include a mode indicator indicative of whether the instrument 50 is usable in a training mode.

If the instrument 50 is usable in a training mode, then the controller 21a outputs a message indicating this at step 112 on any of the screens 32, 34, etc. and enables the actuation of the instrument 50 by the robotic arm 40, IDU 52, etc. In training mode, the system 10 is configured to minimize the mechanical power imparted by the instrument 50. This may include operating the IDU 52 at a lower current, lower torque thresholds, and the like.

Returning to step 108 where the controller 21a determines whether the system 10 is in a training mode, if the system 10 is not in a training mode, then the controller 21a proceeds to step 114. The controller 21a determines whether the system 10 is in a surgical mode, if not, then the controller 21a returns to step 100 to receive the current operational mode of the system 10. If the system 10 is indeed in the surgical mode, then the controller 21a proceeds to determine whether the instrument 50 is in fact usable in the surgical mode at step 116. In particular, the controller 21a compares the usage data to surgical mode threshold or range. If the remaining useful life of the instrument is below the surgical mode threshold (i.e., remaining useful life is too low), then the instrument 50 is not usable in the surgical procedure and the controller 21a proceeds to step 106. The controller 21a outputs a message on one of the screens 32, 34, etc. of the system 10 stating that the instrument 50 is not usable as requested. The controller 21a also prevents use of the instrument 50 by blocking any user inputs or actions that would activate or pair the instrument 50 to the IDU 52 and the system 10 at large.

By way of an example, if the surgical mode threshold is 80% and the instrument 50 has 90% of useful life left, then the instrument 50 is useful in the surgical mode. Conversely, if the instrument 50 has only 70% of useful life left, then the instrument 50 is not usable in the surgical mode. In further embodiments, the instrument 50 may include a mode indicator indicative of whether the instrument 50 is usable in a surgical mode. Thus, if the indicator indicates that the instrument 50 is usable in the selected mode, the controller 21a proceeds to step 118. If the instrument 50 is usable in a surgical mode, e.g., then the controller 21a outputs a message indicating the same at step 118 on any of the screens 32, 34, etc., and enables the actuation of the instrument 50 by the robotic arm 40, IDU 52, etc. In the surgical mode, the IDU 52 is configured to apply the full mechanical power capacity of the instrument 50.

After the instrument 50 is used in either the training mode or surgical mode, the controller 21a, at step 120, updates the usage data of the surgical instrument 50, e.g., increments number of activations or uses, total force or torque applied, actual time used, or toggles the mode indicator if the current use of the instrument 50 has surpassed the usage thresholds corresponding to the modes. Thus, an instrument 50 that was used in a surgical mode may still be reused in any of the modes as long as its use did not exceed the designated thresholds. Likewise the instrument 50 that is being used in the training mode, may be reused in subsequent training or demonstration uses, provided the current utilization did not surpass the expiration threshold of the instrument 50.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a surgical instrument;
a robotic arm including an instrument drive unit configured to couple to and to actuate the surgical instrument; and
a controller configured to:
access usage data pertaining the surgical instrument;
select an instrument operational mode for the surgical instrument based the usage data, the instrument operational mode being one of a surgical mode or a training mode; and
enable use of the surgical instrument based on the selected instrument operational mode.

2. The surgical robotic system according to claim 1, wherein in the surgical mode, the controller is further configured to fully power the instrument drive unit.

3. The surgical robotic system according to claim 1, wherein in the training mode the instrument drive unit is partially powered.

4. The surgical robotic system according to claim 1, wherein the controller is further configured to:
determine whether the surgical instrument is expired based on the usage data; and
prevent use of the surgical instrument in response to the determination.

5. The surgical robotic system according to claim 1, wherein the controller is further configured to update the usage data following the use of the surgical instrument in the selected instrument operational mode.

6. The surgical robotic system according to claim 1, wherein the controller is further configured to receive a system operational mode, the system operational mode being one of the surgical mode or the training mode.

7. The surgical robotic system according to claim 6, wherein the controller is further configured to enable use of the surgical instrument based on the selected instrument operational mode and the received system operational mode.

8. The surgical robotic system according to claim 1, wherein the usage data includes at least one of number of activations, number of uses, total force, total torque, time used, or a mode indicator.

9. A surgical robotic system comprising:
a surgical instrument;
a robotic arm including an instrument drive unit configured to couple to and to actuate the surgical instrument; and
a controller configured to:
set a system operational mode of the robotic arm in one of a surgical mode or a demonstration mode;
access usage data pertaining the surgical instrument;
compare the usage data to a usage threshold corresponding to an instrument operational mode, the instrument operational mode being one of the surgical mode or the demonstration mode;
prevent use of the surgical instrument in response to the usage data being below the usage threshold; and
enable use of the surgical instrument in response to:
the usage data being above the usage threshold, and
the system operational mode corresponding to the instrument operational mode.

10. The surgical robotic system according to claim 9, wherein in the surgical mode, the controller is further configured to fully power the instrument drive unit.

11. The surgical robotic system according to claim 9, wherein in the demonstration mode the instrument drive unit is partially powered.

12. The surgical robotic system according to claim 9, wherein the controller is further configured to:
determine whether the surgical instrument is expired based on the usage data; and
prevent use of the surgical instrument in response to the determination.

13. The surgical robotic system according to claim 9, wherein the controller is further configured to update the usage data following the use of the surgical instrument.

14. The surgical robotic system according to claim 9, wherein the usage data includes at least one of number of activations, number of uses, total force, total torque, time used, or a mode indicator.

15. A method for enabling use of a surgical instrument, the method comprising:
accessing usage data of the surgical instrument;
comparing the usage data to a usage threshold corresponding to an instrument operational mode of a surgical robotic system, the instrument operational mode being one of a surgical mode or a training mode;
preventing use of the surgical instrument in response to the usage data being below the usage threshold; and
enabling operation of the surgical instrument by an instrument drive unit in response to the usage data being above the usage threshold.

16. The method according to claim 15, further comprising:
operating the surgical instrument in the surgical mode by fully powering the instrument drive unit.

17. The method according to claim 15, further comprising:
operating the surgical instrument in the training mode by partially powering the instrument drive unit.

18. The method according to claim 15, further comprising:
determining whether the surgical instrument is expired based on the usage; and
preventing use of the surgical instrument in response to the determination.

19. The method according to claim 15, further comprising:
updating the usage data following the use of the surgical instrument.

20. The method according to claim 15, wherein accessing usage data includes reading at least one of number of activations, number of uses, total force, total torque, time used, or a mode indicator.
